(19) United States
(12) Patent Application Publication
(10) Pub. No.: US 2009/0130771 A1
(43) Pub. Date: May 21, 2009

(54) Assay device and method

(75) Inventors: Oliver William Hardwicke Davies, Inverness (GB); David Kinniburgh Lang, Stirling (GB); John William Dilleen, Clackmannanshire (GB); Phillip Lowe, Tullibody (GB); Steven Howell, Perthshire (GB); Christopher John Slevin, Edinburgh (GB)

Correspondence Address: STEPTOE & JOHNSON LLP, 1330 CONNECTICUT AVENUE, N.W., WASHINGTON, DC, 20036, US

(73) Assignee: Inverness Medical Switzerland GMBH, Zug (CH)

(21) Appl. No.: 11/988,595

(22) PCT Filed: Jul. 19, 2006
(86) PCT No.: PCT/IB2006/001985

§ 371 Date: Oct. 28, 2008

Related U.S. Application Data
(60) Provisional application No. 60/700,728, filed on Jul. 20, 2005.

(51) Int. Cl. G01N 27/00; B01J 19/00; G01N 21/00; C12M 1/00

(52) U.S. Cl. 436/149; 422/68.1; 435/287.1; 422/82.01; 422/82.05

(57) ABSTRACT

An assay device includes a first reagent including a magnetic particle and a second reagent including detectable component. The first and second reagent can each independently bind to an analyte in a sample. Applying a magnetic field can selectively concentrate the detectable component in a detection zone, where a detectable change ca be measured and related to the amount of analyte in the sample.

### CLAIM OF PRIORITY

This application claims priority to U.S. application Ser. No. 60/700,728, filed Jul. 20, 2005.

### TECHNICAL FIELD

This invention relates to an assay device and method.

### BACKGROUND

A system for measuring a biological sample can use a replaceable cartridge or test strip and a reader. The cartridge accepts a sample and includes one or more reagents for producing a detectable change in the test sample. The detectable change can be related to the amount of an analyte in the sample. The cartridge reader can measure the detectable change and communicate a result to the user. The cartridge reader can calculate the amount of analyte in the sample (e.g. as a concentration of analyte in a liquid sample).

The system can be used by users who need to frequently measure an analyte. In particular, the system can be useful for patients with a chronic condition that requires monitoring. In order to encourage patient compliance with a monitoring regimen, it can be desirable for the system to require a small volume of sample and for the replaceable cartridges to be inexpensive.

### SUMMARY

An assay system can include a replaceable assay device and an assay device reader. The assay device can take the form of a cartridge or test strip. The system can provide high sensitivity, low volume detection of an analyte in a sample. The assay device can be simple to manufacture. Because the assay device can be used only once, low manufacturing costs can be important. The assay device can be supplied with a reagent linked to a magnetic particle, allowing magnetic separation of bound and free label. The label can be detected by optical or electrochemical methods. The system can be simple for patients to use in the home.

In one aspect, an assay device for measuring an analyte in a sample includes a sample chamber including a detection zone, a first reagent having an affinity for the analyte and including a magnetic particle, and a second reagent having an affinity for the analyte and including a detectable component. The first and second reagents are each independently capable of binding to the analyte. The detectable component can be directly detectable, or can be capable of creating a detectable change in the sample.

The detectable component can include an enzyme. The assay device can include a substrate of the enzyme. The detection zone can include an electrode or a plurality of electrodes. The detectable component can be capable of producing an electrically detectable change. The detectable component can include an enzyme capable of catalyzing an oxidation-reduction reaction. The assay device can include a redox mediator disposed in the sample chamber. The enzyme can be a glucose oxidase.

The detection zone can be proximate to a transparent region of the sample chamber. The detectable component can be capable of producing an optically detectable change. The sample chamber can include a reference zone.

The first reagent can include an antibody having an affinity for the analyte. The second reagent can include an antibody having an affinity for the analyte. The antibody of the first reagent can have an affinity for a different epitope of the analyte than the antibody of the second reagent. The assay device can include a magnetic field source proximate to the sample chamber.

In another aspect, a system for measuring an analyte in a sample includes an assay device and an assay device reader. The assay device includes a sample chamber including a detection zone, a first reagent having an affinity for the analyte and including a magnetic particle, and a second reagent having an affinity for the analyte and including a detectable component. The first reagent and the second reagent are each independently capable of binding to the analyte. The system includes a magnetic field source configured to selectively apply a magnetic field proximate to the sample chamber. The assay device of the system may be an assay device as described above.

The magnetic field can be selected to move the magnetic particle to the detection zone. The assay device reader can be configured to measure a detectable change at the detection zone. The assay device reader can measure the detectable change at the detection zone while the magnetic field source is applying the magnetic field proximate to the sample chamber. The assay device reader can measure the detectable change at the detection zone while the magnetic field source is substantially not applying the magnetic field proximate to the sample chamber.

The detection zone can include an electrode or a plurality of electrodes. The detectable component can be capable of producing an electrically detectable change. The assay device reader can be in electrical communication with the electrode.

In another aspect, a method of measuring an analyte in a sample includes applying the sample to an assay device including a sample chamber including a detection zone, and applying a magnetic field proximate to the sample chamber. The assay device also includes a first reagent having an affinity for the analyte and including a magnetic particle, and a second reagent having an affinity for the analyte and including a detectable component. The first reagent and the second reagent are each independently capable of binding to the analyte. The method can include allowing a predetermined period of time to pass before applying the magnetic field, thereby allowing the first reagent, the second reagent, or both, to bind the analyte. The assay device used in the method may be the device as described above.

When both reagents are bound to the analyte via different epitopes, the resultant complex can be moved in a magnetic field. If, however, the second reagent is not bound to the first reagent via the analyte, the second reagent will not move in a magnetic field. Thus it is possible to separate the fraction of second reagent bound to the first reagent via the analyte from the unbound fraction of second reagent. In this way it is possible to present an amount of the detectable component to the detection zone that is proportional to the concentration of analyte in the test sample.

The method can include measuring a detectable change at the detection zone. The method can include introducing the assay device to an assay device reader, wherein the assay device reader includes a magnetic field source configured to apply a magnetic field proximate to the sample chamber. The assay device reader can be configured to measure the detectable change at the detection zone.

In another aspect, a device for measuring an analyte in a sample includes a sample chamber including a detection zone, a magnetic field source proximate to the detection zone, and an electrode within the detection zone. The device can include a plurality of electrodes.

In another aspect, a method for measuring an analyte in a sample includes applying the sample to an assay device including a sample chamber including a detection zone, a first reagent having an affinity for the analyte and including a magnetic particle, and a second reagent having an affinity for the analyte and including a detectable component. The method includes applying a magnetic field proximate to the sample chamber, the magnetic field being effective to move the magnetic particle to the detection zone, and detecting the detectable component.

The details of one or more embodiments are set forth in the drawings and description below. Other features, objects, and advantages will be apparent from the description, the drawings, and from the claims.

### BRIEF DESCRIPTION OF THE DRAWING

FIG. 1 is a schematic top view of an assay device base.

FIG. 2 is a schematic end view of an assembled assay device.

FIGS. 3A-3C are schematic cross sections along line III-III of FIG. 1.

FIGS. 4A-4B are schematic depictions of reagents and analytes.

FIGS. 5A-5B are schematic side views of an assay device in operation.

FIG. 6 is an illustration of an assay device reader.

### DETAILED DESCRIPTION

In general, an assay device (e.g., a cartridge or test strip) includes a base and a lid. A void between the base and lid defines a reaction cell which defines the assay volume. The reaction cell is adapted to hold a sample for measurement. The base or lid can have projections that form walls defining the assay volume in an assembled assay device. Alternatively, a third component between the base and lid can provide walls to define the void. The assay device includes a sample inlet that can accept a sample for testing. The sample inlet is fluidly connected by a flow path to the assay volume, so as to deliver a fluid sample from the inlet to the assay volume.

The assay device can include, on a surface of the base, lid, or both, at least one reagent zone, a reference zone, a detection zone, or a combination of these. In some embodiments, the assay device includes a plurality of reagent zones, a reference zone and a detection zone. The reagent zones can overlap with one another or with the reference or detection zones; or the reagent zones can be separated from each other or from the reference and detection zones. Typically the reference and detection zones will be separated from each other. The detection zone and reference zone can be located such that a sample in the assay volume contacts the detection zone and reference zone. A reagent zone can be located such that a sample will contact the reagent zone after the sample is applied to the sample inlet. For example, the reagent zone can be on the flow path, or in the assay volume.

At least one reagent zone includes a first reagent capable of recognizing a desired analyte. Recognition can include binding the analyte. For example, recognition includes selectively binding the analyte; that is, binding the analyte with a higher affinity than other components in the sample. This recognition reagent can be, for example, a protein, a peptide, an antibody, a nucleic acid, a small molecule, a modified antibody, a chimeric antibody, a soluble receptor, an aptamer, or other species capable of binding the analyte. The recognition reagent is optionally linked (e.g., by covalent bond, electrostatic interaction, adsorption, or other chemical or physical linkage) to a reagent that can produce a detectable change. The detectable change can be, for example, a change in optical properties (e.g., a change in absorption, reflectance, refraction, transmittance, or emission of light), or electrical properties (e.g., redox potential, a voltage, a current, or the like).

A reagent zone can include a second reagent capable of recognizing a desired analyte. The second reagent can recognize the same or a different analyte. The first and second recognition reagents can be selected to recognize the same analyte simultaneously. For example the first and second recognition reagents can each be an antibody that recognizes distinct epitopes of the analyte. In this way, a ternary (i.e., three-component) complex of analyte, first recognition reagent and second recognition reagent can be formed. In general, the first and second recognition reagents do not associate with one another in the absence of analyte. The presence of analyte, however, can associate the first and second recognition reagents together, in a ternary complex.

The second recognition reagent can be linked to a surface or to a reagent that can produce a detectable change. The surface can be, for example, a surface of the assay device base, or a surface of a particle. The particle can be, for example, a polymer microsphere, a metal nanoparticle, or a magnetic particle. A magnetic particle is a particle that is influenced by a magnetic field. The magnetic particle can be, for example, a magnetic particle described, in U.S. Patent Application Publication Nos. 20050147963 or 20050100930, or U.S. Pat. No. 5,348,876, each of which is incorporated by reference in its entirety, or commercially available beads, for example, those produced by Dynal AS under the trade name DYNABEADS . Description of recognition reagents linked to surfaces are described in, for example, U.S. Pat. Nos. 6,682,648 and 6,406,913, each of which is incorporated by reference in its entirety. In particular, antibodies linked to magnetic particles are described in, for example, U.S. Patent Application Nos. 20050149169, 20050148096, 20050142549, 20050074748, 20050148096, 20050106652, and 20050100930, and U.S. Pat. No. 5,348,876, which is incorporated by reference in its entirety.

Generally, the detection zone collects the analyte and is the site of a detectable change. The extent of detectable change can be measured at the detection zone. Usually, greater amounts of analyte will result in a greater detectable change; however, the assay can also be configured to produce a smaller change when the analyte is present in greater quantities. The detection zone can collect the analyte by immobilizing it (for example, with a reagent immobilized in the detection zone, where the immobilized reagent binds to the analyte). Alternatively, the detection zone can attract or immobilize a component associated with the analyte. For example, a recognition reagent that binds the analyte and is linked to a magnetic particle can be attracted to the detection zone by a magnetic field provided in the detection zone.

In some embodiments, the detection zone includes an electrode, or a plurality of electrodes. The electrode can be formed of a material selected for electrical conductivity and low reactivity with sample components, for example, silver, gold, aluminum, palladium, platinum, iridium, a conductive carbon, a doped tin oxide, stainless steel, or a conductive polymer. The electrode in the detection zone (the working electrode), in conjunction with a second electrode in a reference zone (the reference electrode) can measure an electrical property of the sample, such as a voltage or a current. Assay devices including electrodes for measuring electrical properties of a sample are described in, for example, U.S. Pat. Nos. 5,708,247, 6,241,862, and 6,733,655, each of which is incorporated by reference in its entirety.

In some embodiments, the assay device base, assay device lid, or both have a translucent or transparent window aligned with the detection zone. An optical change that occurs in the detection zone can be detected through the window. Detection can be done visually (i.e., the change is measured by the user's eye) or measured by an instrument (e.g., a photodiode, photomultiplier, or the like).

In general, the reference zone is similar in nature to the detection zone. In other words, when the detection zone includes an electrode, the reference can likewise include an electrode. When the detection zone is aligned with a window for optical measurement, the reference zone can similarly be aligned with a window for optical measurement. In contrast to the detection zone, the reference zone is not configured to collect analyte. Thus, the detectable change measured in the reference zone can be considered a background measurement to be accounted for when determining the amount of analyte present in the sample.

The sample can be any biological fluid, such as, for example, blood, blood plasma, serum, urine, saliva, tears, or other bodily fluid. The analyte can be any component that is found (or may potentially be found) in the sample, such as, for example, a protein, a peptide, a nucleic acid, a metabolite, a saccharide or polysaccharide, a lipid, a drug or drug metabolite, or other component. The assay device can optionally be supplied with a blood separation membrane arranged between a sample inlet and the detection zone, such that when whole blood is available as a sample, only blood plasma reaches the detection zone.

The assay device and included reagents are typically provided in a dry state. Addition of a liquid sample to the assay device (i.e., to the assay volume) can resuspend dry reagents.

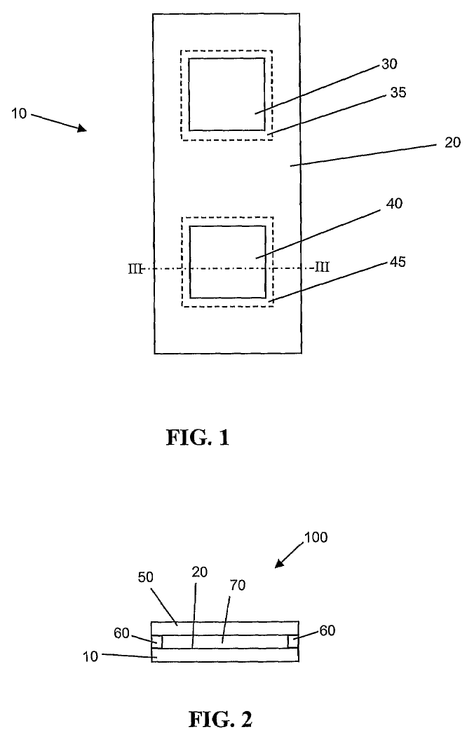

Referring to FIG. 1, assay device base 10 of an assay device includes surface 20. Detection zone 30 and reference zone 40 are disposed on surface 20. First reagent zone 35 overlaps detection zone 30, and second reagent zone 45 overlaps reference zone 40.

In one embodiment, detection zone 30 includes a working electrode, and reference zone 40 includes a reference electrode. First reagent zone 35 includes a redox active enzyme substrate (e.g., glucose) and a redox mediator (e.g., potassium ferricyanide, K₃Fe(CN)₆). Second reagent zone 45 includes a first recognition reagent selected to bind a desired analyte. The first recognition reagent is linked to an enzyme capable of oxidizing or reducing the redox active enzyme substrate. For example, when the redox active enzyme substrate is glucose, the enzyme can be a glucose oxidase (GOD). Second reagent zone 45 can further include a second recognition reagent selected to bind the desired analyte. In particular, the second recognition reagent is selected to bind the desired analyte simultaneously with the first recognition reagent to form a ternary complex.

Referring to FIG. 2, assembled assay device 100 includes base 10 separated from lid 50 by spacers 60. Spacers 60 can be formed as an integral part of base 10 or lid 50. Alternatively, base 10, lid 50 and spacers 60 can be formed separately and assembled together. When assembled, together, connections between base 10, lid 50 and spacers 60 can be sealed, for example with an adhesive or by welding. Base 10, lid 50 and spacers 60 can define a liquid-tight volume 70 where a liquid sample is allowed to contact interior surfaces of volume 70, such as surface 20 of base 10. The dimensions of spacer 60 can be selected such that surfaces of base 10 and lid 50 facing the interior of volume 70 form a capillary, i.e., the base and lid provide capillary action to a liquid inside volume 70. Alternatively, base 10 or lid 50 can provide capillary action independently of each other. Volume 70 can have a volume of less than 100 microliters, less than 20 microliters, less than 10 microliters, or 5 microliters or less.

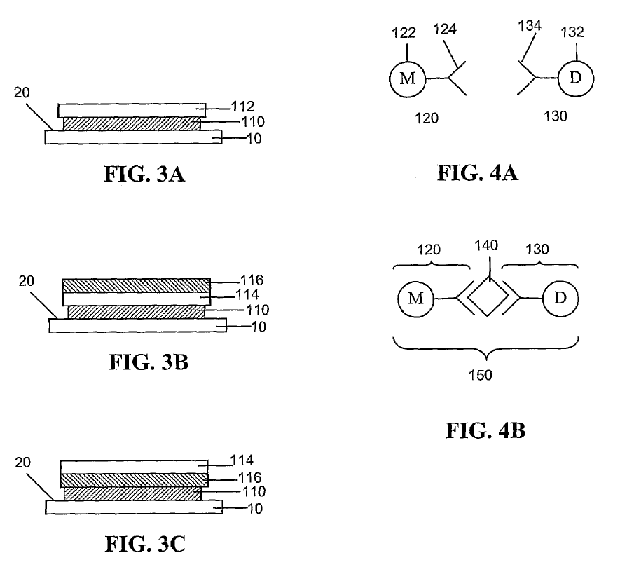

FIG. 3 illustrates alternate configurations of reagent deposition on base 10, as a cross-section along line III-III in FIG. 1. In FIG. 3A, electrode 110 is arranged on surface 20 of base 10. Reagent mixture 112 is deposited over electrode 110. Reagent mixture 112 includes reagent 120 and 130, illustrated in FIG. 4A. Reagent 120 includes magnetic particle 122 linked to antibody 124. Reagent 130 includes detectable component 132 linked to antibody 134. An alternate configuration is shown in FIG. 3B, in which electrode 110 is arranged on surface 20 of base 10, overlayed with reagent mixture 114, which in turn is overlayed with reagent mixture 116. Reagent mixture 114 includes reagent 130, and reagent mixture 116 includes reagent 120. Alternatively, as shown in FIG. 3C, the order of reagent mixtures 114 and 116 can be reversed. Selecting the order in which reagents are deposited can allow selective or timed release of the reagent upon contact with a sample, in order to suit assay kinetics and improve sensitivity.

When a sample is introduced to volume 70, (for example, by contacting the sample with a sample inlet), liquid can fill volume 70 and contact surface 20 of base 10, resuspending the reagents deposited on surface 20. If the sample contains the analyte recognized by antibodies 124 and 134, then the antibodies will bind to the analyte. The antibodies are chosen to bind to different epitopes of the analyte, allowing the formation of a ternary complex 150 of reagent 120, analyte 140, and reagent 130, as illustrated in FIG. 4B.

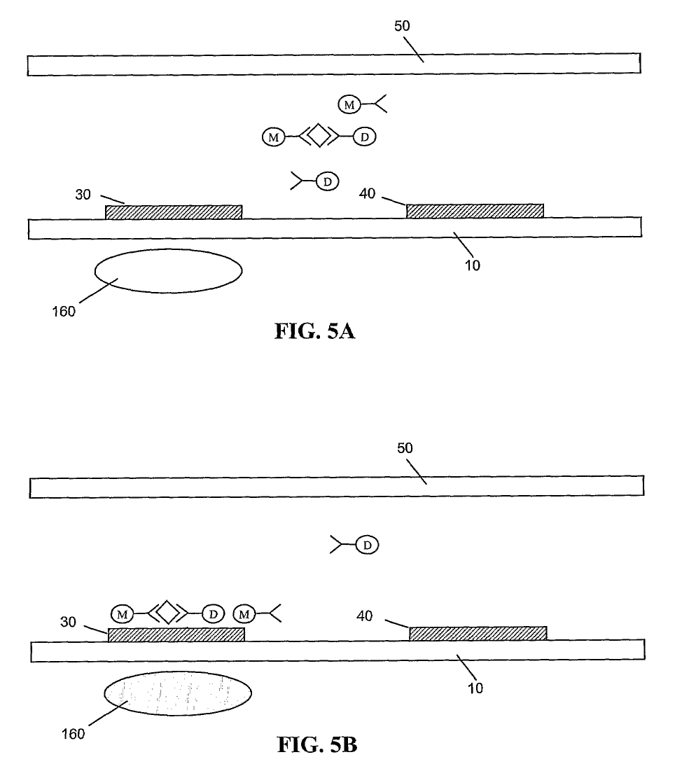

FIGS. 5A and 5B illustrate the assay device, for example, cartridge or test strip, during operation. In FIG. 5A, a side view into volume 70, base 10 and lid 50 confine a liquid sample which includes dissolved reagents and analyte. The reagents can be supplied in excess relative to the amount of analyte present in the sample, such that all analyte is bound, while a portion of the reagents can remain unbound. After the sample is introduced to the assay device, reagents are resuspended by the sample. Reagents, analytes, and complexes can be distributed by diffusion near the location in volume 70 where the reagents originated. As such, no species is localized near detection zone 30, nor near reference zone 40. Magnetic field source 160 is located proximate to detection zone 30. FIG. 5A illustrates the device when source 160 is not applying a magnetic field.

The magnetic field source can be configured to provide a shaped magnetic field. A shaped magnetic field can have magnetic field lines designed to direct magnetic particles toward the detection zone. Such a shaped magnetic field can be useful to control the diffusion or migration of magnetic particles and label particles. More than one magnetic field source can be provided, particularly when a shaped magnetic field is desired. For example, magnetic field sources can be provided at either end of an assay device, where one is configured to attract magnetic particles and the other to repel magnetic particles. Such a configuration can favor the location of all magnetic particles at one end of the assay device.

Detectable component 132 can be directly detectable (e.g., a colored particle detected by observation of a color change, or component 132 can be detected indirectly. Component 132 can produce a product that is directly detected, such that detection of the product is an indirect detection of component 132. For example, component 132 can be an enzyme whose product is detected directly (e.g., optically or electrochemically). The amount of product formed, or rate of product formation, can be related to the amount of detectable component 132.

Glucose oxidase (GOD) is one enzyme that can be used as detectable component 132. In the presence of glucose and mediator, the GOD (whether or not the associated particle is bound to a magnetic particle via the analyte) converts glucose to gluconic acid and converts the mediator (e.g., ferricyanide) from an oxidized form to a reduced from. GOD particles will be substantially absent from detection zone 30 unless a magnetic field selected to move magnetic particles (and GOD particles associated via analyte 140) to detection zone 30 has been applied. After a predetermined period of time has elapsed to allow formation of ternary complex 150, a working electrode in detection zone 30 can be turned on. The amount of reduced mediator in the bulk fluid is measured as a current at the working electrode or electrodes. This current, produced when the GOD is distributed homogeneously in the sample, is the background signal.

When magnetic field source 160 applies a magnetic field in the vicinity of detection zone 30 (see FIG. 5B), magnetic particles of reagent 120 become localized near detection zone 30. The magnetic field localizes particles whether the particles are bound to reagent or not. The unbound portion of reagent 130 remains distributed throughout volume 70. Thus, application of a magnetic field by source 160 causes an increase in the concentration of enzyme 132 near detection zone 30. Enzyme 132 in turn produces a change detectable in detection zone 30.

When enzyme 132 is GOD, the increased concentration of reduced mediator at the surface of working electrode 30 is reflected as a higher current at that electrode when the magnetic field is applied. The higher the analyte concentration, the larger the current will be.

The magnetic field can be applied and removed a number of times, and a series of magnetized and non-magnetized working electrode currents can be measured. The data collected allow the concentration of analyte in the sample to be measured.

In some embodiments, two working electrodes can be used, one with a magnet and one without, each on opposite internal faces of volume 70. In this case, one electrode is magnetized while the other is not, and both electrodes are activated simultaneously. The currents at the two working electrodes are then compared.

Detectable component 132 can be selected to produce an optical change. For example, a detectable change in chemiluminescent signal can be produced when an analyte molecule in a sample brings two particles (or beads) together in close proximity. A first particle, called a donor particle, is linked to a first antibody, and a second particle (an acceptor particle) is linked to a second antibody. The first and second antibodies bind to different epitopes of the same antigen, such that a ternary complex of donor particle-antigen-acceptor particle can be formed. A cascade of chemical reactions that depends on the proximity of the beads (and therefore on the presence of the analyte) can produce greatly amplified signal. Detection of an analyte at attomolar (i.e., on the order of 10-18 molar) concentrations is possible.

Photosensitizer particles (donor particles) including a phthalocyanine can generate singlet oxygen when irradiated with light having a wavelength of 680 nm. The singlet oxygen produced has a very short half-life—about 4 microseconds—and hence it decays rapidly to a ground state. Because of the short half-life, singlet oxygen can only diffuse to a distance of a few hundred microns from the surface of the particles before it decays to ground state. The singlet state survives long enough, however, to enter a second particle held in close proximity. The second particles (acceptor particles) include a dye that is activated by singlet oxygen to produce chemiluminescent emission. This chemiluminescent emission can activate further fluorophores contained in the same particle, subsequently causing emission of light at 520-620 nm. See, for example, Proc. Natl. Acad. Sci. 91:5426-5430 1994; and U.S. Pat. No. 6,143,514, each of which is incorporated by reference in its entirety.

An optical change can also be produced by a bead linked to an antibody. The bead can include a polymeric material, for example, latex or polystyrene. To produce the optical change, the bead can include a light-absorbing or light-emitting compound. For example, a latex bead can include a dye or a fluorescent compound. The reagent can include a plurality of beads. The beads in the plurality can be linked to one or more distinct antibodies. A single bead can be linked to two or more distinct antibodies, or each bead can have only one distinct antibody linked to it. The reagent can have more than one distinct antibody each capable of binding to the same analyte, or antibodies that recognizes different analytes. When the bead includes a light absorbing compound, the optical measurement can be a measurement of transmittance, absorbance or reflectance. With a fluorescent compound, the intensity of emitted light can be measured. The extent of the measured optical change can be correlated to the concentration of analyte in the sample.

A detectable change can be produced by the enzyme multiplied immunoassay technique (EMIT). In an EMIT assay format, an enzyme-analyte conjugate is used. A first reagent can include an antibody specific for the analyte, an enzyme substrate, and (optionally) a coenzyme. A second reagent can include a labeled analyte: a modified analyte that is linked to an enzyme. For example, the enzyme can be a glucose-6-phosphate dehydrogenase (G-6-PDH). G-6-PDH can catalyze the reaction of glucose-6-phosphate with NAD(P) to yield 6-phosphoglucono-D-lactone and NAD(P)H. NAD(P)H absorbs light with a wavelength of 340 nm, whereas NAD(P) does not. Thus, a change in absorption of 340 nm light as a result of the G-6-PDH catalyzed reaction can be a detectable change. When the first reagent is mixed with a sample, the analyte is bound by the antibody in the first reagent. The second reagent is added, and any free antibody binding sites are occupied by the enzyme-linked analyte of the second reagent. Any remaining free antibodies bind the labeled analyte, inactivating the linked enzyme. Labeled analyte bound by the antibody is inactive, i.e., it does not contribute to the detectable change. Labeled analyte that is not bound by antibody (a quantity proportional to amount of analyte in sample) reacts with the substrate to form a detectable product (e.g., NAD(P)H).

Another assay format is the cloned enzyme donor immunoassay (CEDIA). CEDIA is a homogeneous immunoassay based on the bacterial enzyme β-galactosidase of E. coli which has been genetically engineered into two inactive fragments. These two inactive fragments can recombine to form an active enzyme. One fragment consists of an analyte-fragment conjugate, and the other consists of an antibody-fragment conjugate. The amount of active enzyme that generates the signal is proportional to the analyte concentration. See, for example, Khanna, P. L. and Coty, W. A. (1993) In: Methods of Immunological Analysis, volume 1 (Masseyeff, R. F., Albert, W. H., and Staines, N. A., eds.) Weinheim, FRG: VCH Verlagsgesellschaft MbH, 1993: 416-426; Coty, W. A., Loor, R., Powell, M., and Khanna, P. L. (1994) J. Clin. Immunoassay 17(3): 144-150; and Coty, W. A., Shindelman, J., Rouhani, R. and Powell, M. J. (1999) Genetic Engineering News 19(7), each of which is incorporated by reference in its entirety.

The assay device can be used in combination with a reader configured to measure the detectable change. The reader can include an optical system to detect light from the analysis region. The light to be detected can be, for example, emitted, transmitted, reflected, or scattered from the detection zone. Emitted light can result from, for example, chemiluminescent or fluorescent emission. The optical system can include an illumination source, for example, to be used in the detection of a change in fluorescence, absorbance, or reflection of light. For an assay device configured for an electrochemical measurement, the reader can be in electrical contact with the working electrode and reference electrode. The assay device electrodes can have electrical leads connecting the electrodes to contacts outside the assay void. The contacts register with and contact corresponding contacts of the assay device to provide electrical contact. The reader can also include an output display configured to display the results of the measurement to a user.

The assay device reader can include magnetic field source 160. The assay device reader can be configured to apply a magnetic field via source 160 at predetermined times, such as after a predetermined period of time has elapsed after a sample has been applied to the assay device. Magnetic field source 160 can be, for example, an electromagnet or a permanent magnet. An electromagnet can selectively apply a field when a current is supplied to the electromagnet. A permanent magnet can be moved toward or away from the detection zone in order to control the strength of the field at that site.

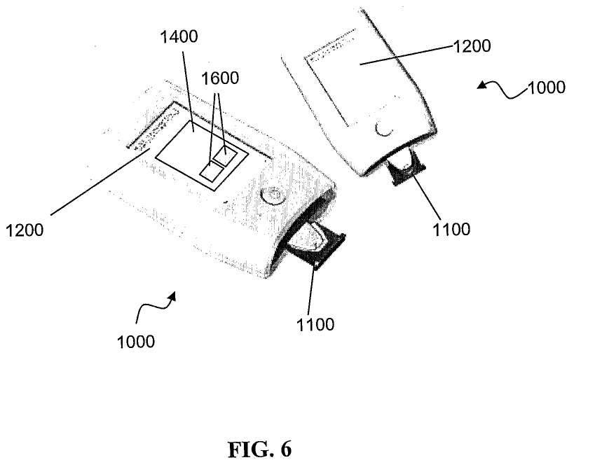

Referring to FIG. 6, reader instrument 1000 accepts test assay device 1100 and includes display 1200. The display 1200 may be used to display images in various formats, for example, text, joint photographic experts group (JPEG) format, tagged image file format (TIFF), graphics interchange format (GIF), or bitmap. Display 1200 can also be used to display text messages, help messages, instructions, queries, test results, and various information to patients.

Display 1200 can provide a user with an input region 1400. Input region 1400 can include keys 1600. In one embodiment, input region 1400 can be implemented as symbols displayed on the display 1200, for example when display 1200 is a touch-sensitive screen. User instructions and queries are presented to the user on display 1200. The user can respond to the queries via the input region.

Reader 1000 also includes an assay device reader, which accepts diagnostic test assay devices 1100 for reading. The assay device reader can measure the level of an analyte based on, for example, the magnitude of an optical change, an electrical change, or other detectable change that occurs on a test assay device 1100. For reading assay devices that produce an optical change in response to analyte, the assay device reader can include optical systems for measuring the detectable change, for example, a light source, filter, and photon detector, e.g., a photodiode, photomultiplier, or Avalance photo diode. For reading assay devices that produce an electrical change in response to analyte, the assay device reader can include electrical systems for measuring the detectable change, including, for example, a voltameter or amperometer.

Device 1000 further can include a communication port (not pictured). The communication port can be, for example, a connection to a telephone line or computer network. Device 1000 can communicate the results of a measurement to an output device, remote computer, or to a health care provider from a remote location.

A patient, health care provider, or other user can use reader 1000 for testing and recording the levels of various analytes, such as, for example, a biomarker, a metabolite, or a drug of abuse. Various implementations of diagnostic device 1000 may access programs and/or data stored on a storage medium (e.g., a hard disk drive (HDD), flash memory, video cassette recorder (VCR) tape or digital video disc (DVD); compact disc (CD); or floppy disk). Additionally, various implementations may access programs and/or data accessed stored on another computer system through a communication medium including a direct cable connection, a computer network, a wireless network, a satellite network, or the like.

The software controlling the reader can be in the form of a software application running on any processing device, such as, a general-purpose computing device, a personal digital assistant (PDA), a special-purpose computing device, a laptop computer, a handheld computer, or a network appliance.

The reader may be implemented using a hardware configuration including a processor, one or more input devices, one or more output devices, a computer-readable medium, and a computer memory device. The processor may be implemented using any computer processing device, such as, a general-purpose microprocessor or an application-specific integrated circuit (ASIC). The processor can be integrated with input/output (I/O) devices to provide a mechanism to receive sensor data and/or input data and to provide a mechanism to display or otherwise output queries and results to a service technician. Input device may include, for example, one or more of the following: a mouse, a keyboard, a touch-screen display, a button, a sensor, and a counter.

The display 1200 may be implemented using any output technology, including a liquid crystal display (LCD), a television, a printer, and a light emitting diode (LED). The computer-readable medium provides a mechanism for storing programs and data either on a fixed or removable medium. The computer-readable medium may be implemented using a conventional computer hard drive, or other removable medium. Finally, the system uses a computer memory device, such as a random access memory (RAM), to assist in operating the reader.

Implementations of the reader can include software that directs the user in using the device, stores the results of measurements. The reader 1000 can provide access to applications such as a medical records database or other systems used in the care of patients. In one example, the device connects to a medical records database via the communication port. Device 1000 may also have the ability to go online, integrating existing databases and linking other websites.

In general, the assay device can be made by depositing reagents on a base and sealing a lid over the base. The base can be a micro-molded platform or a laminate platform.

Micro-molded Platform

For an assay device prepared for optical detection, the base, the lid, or both base and lid can be transparent to a desired wavelength of light. Typically both base and lid are transparent to visible wavelengths of light, e.g., 400-700 nm. The base and lid can be transparent to near UV and near IR wavelengths, for example, to provide a range of wavelengths that can be used for detection, such as 200 nm to 1000 nm, or 300 nm to 900 nm.

For an assay device that will use electrochemical detection, electrodes are deposited on a surface of the base. The electrodes can be deposited by screen printing on the base with a carbon or silver ink, followed by an insulation ink; by evaporation or sputtering of a conductive material (such as, for example, gold, silver or aluminum) on the base, followed by laser ablation; or evaporation or sputtering of a conductive material (such as, for example, gold, silver or aluminum) on the base, followed by photolithographic masking and a wet or dry etch.

An electrode can be formed on the lid in one of two ways. A rigid lid can be prepared with one or more through holes, mounted to a vacuum base, and screen printing used to deposit carbon or silver ink. Drawing a vacuum on the underside of the rigid lid while screen printing draws the conductive ink into the through holes, creating electrical contact between the topside and underside of the lid, and sealing the hole to ensure that no liquid can leak out. Alternatively, the lid can be manufactured without any through holes and placed, inverted, on a screen printing platform, where carbon or silver ink is printed.

Once the electrodes have been prepared, the micro-molded bases are loaded and registered to a known location for reagent deposition. Deposition of reagents can be accomplished by dispensing or aspirating from a nozzle, using an electromagnetic valve and servo- or stepper-driven syringe. These methods can deposit droplets or lines of reagents in a contact or non-contact mode. Other methods for depositing reagents include pad printing, screen printing, piezoelectric print head (e.g., ink-jet printing), or depositing from a pouch which is compressed to release reagent (a “cake icer”). Deposition can preferably be performed in a humidity- and temperature-controlled environment. Different reagents can be dispensed at the same or at a different station.

Fluorescent or colored additives can optionally be added to the reagents to allow detection of cross contamination or overspill of the reagents outside the desired deposition zone. Product performance can be impaired by cross-contamination. Deposition zones can be in close proximity or a distance apart. The fluorescent or colored additives are selected so as not to interfere with the operation of the assay device, particularly with detection of the analyte.

After deposition, the reagents are dried. Drying can be achieved by ambient air drying, infrared drying, infrared drying assisted by forced air, ultraviolet light drying, forced warm, controlled relative humidity drying, or a combination of these.

Micro-molded bases can then be lidded by bonding a flexible or rigid lid on top. Registration of the base and lid occurs before the two are bonded together. The base and lid can be bonded by heat sealing (using a heat activated adhesive previously applied to lid or base, by ultrasonic welding to join two similar materials, by laser welding (mask or line laser to join two similar materials), by cyanoacrylate adhesive, by epoxy adhesive previously applied to the lid or base, or by a pressure sensitive adhesive previously applied to the lid or base.

After lidding, some or all of the assembled assay devices can be inspected for critical dimensions, to ensure that the assay device will perform as designed. Inspection can include visual inspection, laser inspection, contact measurement, or a combination of these.

The assay device can include a buffer pouch. The buffer pouch can be a molded well having a bottom and a top opening. The lower opening can be sealed with a rupturable foil or plastic, and the well filled with buffer. A stronger foil or laminate is then sealed over the top opening. Alternatively, a preformed blister pouch filled with buffer is placed in and bonded in the well. The blister pouch can include 50 to 200 μL of buffer and is formed, filled, and sealed using standard blister methods. The blister material can be foil or plastic. The blister can be bonded to the well with pressure sensitive adhesive or a cyanoacrylate adhesive.

Laminate Platform

Three or more laminates, fed on a roll form at a specified width, can be used to construct an assay device. The base laminate is a plastic material and is coated on one surface with a hydrophilic material. This laminate is fed into a printing station for deposition of conductive electrodes and insulation inks. The base laminate is registered (cross web) and the conductive electrodes deposited on the hydrophilic surface, by the techniques described previously.

The base laminate is then fed to a deposition station and one or more reagents applied to the laminate. Registration, both cross web and down web, occurs before reagents are deposited by the methods described above. The reagents are dried following deposition by the methods described above.

A middle laminate is fed in roll form at a specified width. There can be more than one middle laminate in an assay device. The term middle serves to indicate that it is not a base laminate or lid laminate. A middle laminate can be a plastic spacer with either a pressure sensitive adhesive or a heat seal adhesive on either face of the laminate. A pressure sensitive adhesive is provided with a protective liner on either side to protect the adhesive. Variations in the thickness of the middle laminate and its adhesives is less than 15%, or less than 10%.

Channels and features are cut into the middle laminate using a laser source (e.g., a CO₂ laser, a YAG laser, an excimer laser, or other). Channels and features can be cut all the way through the thickness of the middle laminate, or the features and channels can be ablated to a controlled depth from one face of the laminate.

The middle and base laminates are registered in both the cross web and down web directions, and bonded together. If a pressure sensitive adhesive is used, the lower liner is removed from the middle laminate and pressure is applied to bond the base to the middle laminate. If a heat seal adhesive is used, the base and middle laminate are bonded using heat and pressure.

The top laminate, which forms the lid of the assay device, is fed in roll form at a specified width. The top laminate can be a plastic material. Features can be cut into the top laminate using a laser source as described above. The top laminate is registered (cross web and down web) to the base and middle laminates, and bonded by pressure lamination or by heat and pressure lamination, depending on the adhesive used.

After the laminate is registered in cross and down web directions, discrete assay devices or test strips are cut from the laminate using a high powered laser (such as, for example, a CO₂ laser, a YAG laser, an excimer laser, or other).

Some or all of the assembled assay devices can be inspected for critical dimensions, to ensure that the assay device will fit perform as designed. Inspection can include visual inspection, laser inspection, contact measurement, or a combination of these.

Other embodiments are within the scope of the following claims.

1. An assay device for measuring an analyte in a sample comprising:
a sample chamber including a detection zone;
a first reagent having an affinity for the analyte and including a magnetic particle; and
a second reagent having an affinity for the analyte and including a detectable component;
wherein the first and second reagents are each independently capable of binding to the analyte.

2. The assay device of claim 1, wherein the detectable component is directly detectable.

3. The assay device of claim 1, wherein the detectable component is capable of creating a detectable change in the sample.

4. The assay device of claim 1, wherein the detectable component includes an enzyme.

5. The assay device of claim 1, wherein the device further includes a substrate of the enzyme.

6. The assay device of claim 1, wherein the detection zone includes an electrode.

7. The assay device of claim 1, wherein the detection zone includes a plurality of electrodes.

8. The assay device of claim 7, wherein the detectable component is capable of producing an electrically detectable change.

9. The assay device of claim 8, wherein the detectable component includes an enzyme capable of catalyzing an oxidation-reduction reaction.

10. The assay device of claim 9, further comprising a redox mediator disposed in the sample chamber.

11. The assay device of claim 10, wherein the enzyme is a glucose oxidase.

12. The assay device of claim 1, wherein the detection zone is proximate to a transparent region of the sample chamber.

13. The assay device of claim 12, wherein the detectable component is capable of producing an optically detectable change.

14. The assay device of claim 1, wherein the sample chamber further includes a reference zone.

15. The assay device of claim 1, wherein the first reagent includes an antibody having an affinity for the analyte.

16. The assay device of claim 1, wherein the second reagent includes an antibody having an affinity for the analyte.

17. The assay device of claim 1, wherein the second reagent includes an antibody having an affinity for the analyte, and the antibody of the first reagent has an affinity for a different epitope of the analyte than the antibody of the second reagent.

18. The assay device of claim 1, further including a magnetic field source proximate to the sample chamber.

19. A system for measuring an analyte in a sample comprising:
an assay device including:
a sample chamber including a detection zone;
a first reagent having an affinity for the analyte and including a magnetic particle; and
a second reagent having an affinity for the analyte and including a detectable component;
wherein the first reagent and the second reagent are each independently capable of binding to the analyte; and
an assay device reader,
wherein the system includes a magnetic field source configured to selectively apply a magnetic field proximate to the sample chamber.

20-37. (canceled)

38. A method of measuring an analyte in a sample comprising:
applying the sample to an assay device including:
a sample chamber including a detection zone;
a first reagent having an affinity for the analyte and including a magnetic particle; and
a second reagent having an affinity for the analyte and including a detectable component;
wherein the first reagent and the second reagent are each independently capable of binding to the analyte; and
applying a magnetic field proximate to the sample chamber.

39-58. (canceled)

59. A device for measuring an analyte in a sample comprising:
a sample chamber including a detection zone;
a magnetic field source proximate to the sample chamber; and
an electrode within the detection zone.

60. The device of claim 59, wherein the detection zone includes a plurality of electrodes.
